(19) United States
(12) Patent Application Publication
(10) Pub. No.: US 2004/0146936 A1
(43) Pub. Date: Jul. 29, 2004

(54) System and method for identifying cellular pathways and interactions

(76) Inventors: Joel S. Bader, Stamford, CT (US); John S. Chant, Branford, CT (US); Cory Brouwer, Guilford, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/602,347

(22) Filed: Jun. 23, 2003

Related U.S. Application Data
(60) Provisional application No. 60/390,763, filed on Jun. 21, 2002.

(51) Int. Cl. G01N 33/53; G06F 19/00; G01N 33/48; G01N 33/50

(52) U.S. Cl. 435/7.1; 702/19

(57) ABSTRACT

The present invention provides a system comprising methods by which the interactions of biological materials can be determined.

### RELATED APPLICATION

This application claims priority to U.S.SNo. 60/390,763, filed Jun. 21, 2002, the contents of which is incorporated herein by reference in its entirety.

### FIELD OF THE INVENTION

The present invention relates to systems and methods that utilize statistical means for analyzing biological samples for cellular interactions including protein-protein interactions (i.e., the present invention is directed in part to methods of identifying a cellular interaction in a biological system, such as protein-protein and protein-nucleic acid interactions).

### BACKGROUND OF THE INVENTION

A challenge of the post-genome era is understanding how individual proteins assemble into complexes and pathways. While proteome-scale experiments have revealed underlying protein-protein interactions, these data sets are dominated by spurious interactions. Genome-scale technologies have always benefited from statistical measures of data quality.

Protein-protein interactions from high-throughput yeast two-hybrid screens (Y2H) (Uetz et al. Nature 403:623 (2000); Ito et al., Proc Natl Acad Sci USA 98: 4569-4574 (2001); Tong et al., Science 295: 321-324 (2002)) and inferred from protein complexes identified by co-immunoprecipitation/mass spectrometry (Co-IP) (Gavin et al., Nature 415: 141-147 (2002); Ho Y et al., Nature 415: 180-183 (2002)) now include about ¾ of the yeast proteome. The intersection of these data sets is only a few percent, implying that the screens are incomplete with 20,000 to 30,000 interactions remaining to be discovered (von Mering et al., Nature 417: 399-403 (2002); Bader et al., Nature Biotech 20: 991-997 (2002)).

A more challenging problem is the prevalence of spurious interactions in the high-throughput data sets. Spurious interactions may arise in the Y2H system from self-activators (Phizicky et al;. Nature 422: 208-215 (2003)), in the Co-IP system from abundant protein contaminants (Aebersold et al, Nature 422: 198-207 (2003)), and in both systems from weak, non-specific interactions with questionable relevance for understanding biological pathways. Previous analysis suggests that only 30-50% of the high-throughput interactions are biologically relevant (Mewes et al., Nucleic Acids Research 30:31-34 (2002); Deane et al., Molecular and Cellular Proteomics 1.5: 349-356 (2002)).

Consequently, a crucial step in analyzing proteomics data is gleaning the subset of credible interactions from the background noise. Three types of methods have been used to separate credible interactions from the background noise: topological criteria (stickiness), intersections between proteomics data sets, and intersections with other types of data.

Topological criteria described in original reports (Uetz et al., Nature 403: 623 (2000); Ito et al., Proc Natl Acad Sci USA 98: 4569-4574 (2001); Gavin et al., Nature 415: 141-147 (2002); Ho Y et al., Nature 415: 180-183 (2002)) and used for later analysis (Maslov et al., Science 296: 910-913 (2002)) have focused on removing sticky proteins, e.g., those with an extremely high number of interaction partners. A difficulty of this method is that the distribution of interactions per protein shows a smooth, power-law decay, with no clear separation between sticky and non-sticky proteins. Furthermore, the scale-free nature of biological networks suggests that highly-connected proteins are a real feature of protein interaction networks (Watts et al., Nature 393: 440 (1998); Barabasi et al., Science 286: 509 (1999); Jeong et al., Nature 411: 41-42 (2001); Ravasz et al., Science 297: 1551-1555 (2002); Wolf et al., Bioessays 24: 105-109 (2002)). A topological approach specific to Co-IP experiments, retaining hub-spoke but not spoke-spoke interactions (Bader et al., Nature Biotech 20: 991-997 (2002), enhances the data quality at the expense of discarding most of the results.

Taking the intersection of multiple high-throughput data sets is known to enrich for credible interactions (von Mering et al., Nature 417: 399-403 (2002); Bader et al., Nature Biotech 20: 991-997 (2002)). A shortcoming of this method is the small number of interactions in the intersection: only 387 pairwise interactions are common to the 6395 Y2H interactions and 41,775 Co-IP interactions considered here. Beyond incompleteness of the screens, other factors reduce the probability that an interaction is observed with both systems: the protein classes amenable to screening; the effective concentrations of the proteins in the engineered Y2H system relative to the in vivo Co-IP system; the strength of the interaction; and the existence of non-direct or stabilized interactions.

Intersections with other types of data are also possible. Interacting proteins whose transcripts are co-expressed are more likely to be credible (Ge et al., Nature Genetics 29: 482-486 (2001); Jansen et al., Genome Research 12: 37-46 (2002)) and have been prioritized for experimental validation (Kemmeren et al., Mol Cell 9: 1133-1143 (2001)). Co-expression is not necessary for transient interactions, and even proteins in a permanent complex may have low transcriptional correlation due to differences in degradation rates (Jansen et al., Genome Research 12: 37-46 (2002)). Co-expression is also not a sufficient criterion, and may actually enrich for spurious interactions. Homology between a pair of proteins and a corresponding pair of interacting proteins has been used to enhance the confidence in high-throughput data (Uetz et al., Nature 403: 623 (2000)), but is necessarily restricted to proteins with known homologs. Even for these proteins, a homology criterion may only identify half of the applicable true interactions as high-confidence (Deane et al., Molecular and Cellular Proteomics 1.5: 349-356 (2002)).

The methods presently known in the art may be suitable for identifying a small, high-confidence set of interactions, but they fail to make strong predictions for the majority of the high-throughput data.

### SUMMARY OF THE INVENTION

The methods according to the present invention are based in part in the discovery that biologically relevant interactions can be identified using predictive interaction models.

In one aspect, the present invention provides for a method of identifying a cellular interaction within a biological system by providing a predictive interaction model that includes one or more training sets, and applying the predictive interaction model to the biological system in order to identify a high-confidence interaction; and thus identifying one or more cellular interactions within the system. The cellular interaction may be a protein-protein interaction, or a protein-nucleic acid interaction. A biological system includes one or more cells or components thereof (e.g., organelles, cell extracts), tissues, organs, organisms, bacteria, viruses, as well as ex vivo and in vitro systems. A high-confidence interaction is generally an interaction with a predicted interaction confidence greater than or equal to 0.5. Alternatively, a high-confidence interaction has a predicted interaction confidence greater than or equal to 0.6, 0.7, 0.8, 0.9, 1.0, 1.5, or 2.0.

In embodiments of the present invention, the method additionally includes validating the identified cellular interaction by comparing it with a control cellular interaction. In related embodiments, the control cellular interaction is an experimentally derived cellular interaction, e.g., one using a yeast two-hybrid system or a co-immunoprecipitation system.

In some embodiments of the present invention, the one or more training sets are positive, negative, non-interacting training sets, or a combination of training sets. In other embodiments, the predictive interaction model may also include one or more explanatory variables, e.g., the co-expression of nucleic acids, the sequence similarity of polypeptides, subcellular localization, or the domain similarity of polypeptides.

In another aspect, the present invention provides for a method of identifying an interacting protein that interacts with a test protein in a biological system, by providing a predictive interaction model including one or more training sets, and applying the predictive interaction model to the biological system to identify a high-confidence interaction between the test and interacting proteins, thus identifying one or more interacting proteins. In such embodiments, the identified interaction may be validated using a yeast two-hybrid system or a co-immunoprecipitation system. The present invention also provides the polypeptide identified by these methods. An interacting protein includes any protein not known to interact with a test protein or portion thereof (either by direct or indirect binding).

In a further aspect, the present invention provides for a method of identifying a compound that modulates a cellular pathway within a biological system by contacting the biological system with a candidate agent such that a high-confidence interaction between a test and interacting protein is modulated, thus identifying a compound that modulates the cellular pathway within the system. In some embodiments, the biological system is a transgenic animal, such as a transgenic mouse. The modulation of a cellular pathway can be, e.g., an increase in gene expression as compared to a control biological system that is not exposed to the candidate agent. The candidate agent can be any chemical or biological compound capable of being isolated or synthesized by one of skill in the art.

In embodiments of the present invention, the agent may increase or decrease the expression of the test protein. The increase or decrease the expression of the test protein is determined relative to a biological system that is not exposed to the candidate agent. In still other embodiments, the agent may increase or decrease the expression of the interacting protein as compared to a biological system that is not exposed to the candidate agent. The present invention also provides the compound identified by these methods.

In another aspect, the present invention provides for a method of identifying a compound that modulates a cellular pathway in a biological system by providing a predictive interaction model including one or more training sets, applying the model to the system to identify a high-confidence interaction between a test and an interacting protein, and contacting the biological system with a candidate agent, such that the high-confidence interaction between the test and the interacting protein is modulated, thereby identifying a compound that modulates the cellular pathway in the biological system.

In another aspect, the present invention provides for a method of diagnosing a test subject with an aberrant cellular pathway by providing a predictive interaction model including one or more training sets, applying the predictive interaction model to a biological sample derived from the test subject to identify a first high-confidence interaction, and comparing the first high-confidence interaction with a second high-confidence interaction derived from a biological sample from a reference subject not affected by an aberrant cellular pathway, whereby a difference in the first and second high-confidence interaction indicates that the test subject has an aberrant cellular pathway. In like manner the present invention also provides for a method of diagnosing a test subject suffering from or at risk of a disease or disorder characterized by an aberrant cellular pathway. An aberrant cellular pathway includes any pathway In such embodiments, the disease may be a cell proliferation-associated disease, a cell differentiation-associated disease, or an apoptosis-associated disease.

The present invention also provides a database including a predictive interaction model that includes one or more training sets.

Unless otherwise defined, all technical and scientific terms used herein have the same meaning as commonly understood by one of ordinary skill in the art to which this invention belongs. Although methods and materials similar or equivalent to those described herein can be used in the practice of the present invention, suitable methods and materials are described below.

Other features and advantages of the invention will be apparent from the following detailed description, and from the claims.

### BRIEF DESCRIPTION OF THE DRAWINGS

FIG. 1 is a block diagram demonstrating pairs of proteins that have been binned according to their shortest path in networks generated from Y2H and Co-IP data. Binning of protein pairs includes the assignment of a location for each protein pair on a two-dimensional grid relative to other protein pairs, according to confidence score. Each bin has a width of 0.1 confidence score units, and the mean and standard error of the mean for each bin is calculated. The false-color map indicates bins with more (light grey) or fewer (dark grey) interactions than expected by chance. Bins are enriched for true-positives, false-positives, and true non-interactors as indicated by ovals.

FIG. 2. is a series of plots depicting protein-protein interactions inferred from Co-IP and Y2H. Networks are binned according to the predicted interaction confidence. For the interactions within each bin, three correlations are computed: the biological process annotation; the cellular component annotation; and the expression correlation from a series of 300 experiments (see, e.g., Example 1). Lines are a guide to the eye only; vertical bars represent the standard error of the mean and, when not visible, are smaller than the size of the point. FIG. 2a is a plot demonstrating annotation correlations tend increase with increasing confidence. There is a break in the slope at a confidence of 0.5, indicating that 0.5 is an appropriate cut between low-confidence (<0.5) and high-confidence (≧0.5) interactions. FIG. 2b is a plot demonstrating that expression correlations for Y2H interactions increase monotonically with interaction confidence. Expression correlations for Co-IP interactions show an increase with both high and low interaction confidence. FIG. 2c is a plot demonstrating the absolute transcriptional level for proteins participating in an interaction is inversely correlated with interaction confidence for interactions identified by Co-IP. There is no significant correlation between transcriptional level and interaction confidence for Y2H interactions.

FIG. 3. is a series of plots depicting properties of the giant component. FIG. 3a is a scatter plot showing that the distribution of distances between pairs of proteins in the physical interaction network shows a peak at 9 degrees of separation for the actual network and 6 for the randomized network. Fitting the data to logistic growth is shown as dashed and dotted lines. The single parameter in the logistic fit is the effective number of interactions per protein (actual network 2.23±0.02, p-value=7×10⁻³⁰; randomized network 4.13±0.06 value=9×10⁻¹⁷). FIG. 3b is a scatter plot showing that the number of loops is much greater for the actual network than the randomized network for loop lengths ≦9. For larger loops in the actual network, and for all loops in the randomized network, the mathematical model provides an accurate fit (actual network 3.10±0.14, p-value=0.026; randomized network 1.3759±0.0003, p-value =1.4×10⁻²⁰). FIG. 3c is a scatter plot showing that correlations calculated from database annotations and expression measurements decline steeply as the distance between a pair of proteins increases to 4 links, followed by a less steep decline. FIG. 3d is a scatter plot demonstrating that genetic interactions are enhanced between proteins up to 4 links apart.

FIG. 4. is a series of schematic illustrations of interaction networks. FIG. 4a uses genetic interactions as anchors to mine the physical interaction network. Proteins have been coloured according to biological process (see Example 1). Connections between proteins having a genetic interactions are colored black (a physical interaction also exists) or medium grey (no direct physical interaction, but separated by 2 or 3 physical interactions). Connections between proteins not having a genetic interactions are colored dark grey (both a Y2H and a Co-IP interaction), medium-light grey (only a Y2H interaction), or light grey (only a Co-IP interaction). Recognizable complexes identified by joint analysis of protein-protein interactions and genetic interactions include the origin recognition complex (FIG. 4b); the actin related protein (ARP) complex (FIG. 4c); the anaphase-promoting complex (FIG. 4d); and the ubiquitin-dependent protein degradation machinery (FIG. 4e). Novel connections between complexes include a connection between the primase complex and the DNA replication factor C complex (FIG. 4f) and connections between the cohesin complex, the spindle pole body, and a microfilament motor protein complex (FIG. 4g).

FIG. 5. is a schematic representation of an expression-anchored interaction network for the cell cycle. Light grey arrows indicate transcripts that are expressed within 10 minutes and proteins are bridged by physical interactions with a third protein; black arrows indicate transcripts that are expressed within 10 minutes and proteins are connected directly by a physical interaction. For proteins whose transcripts are not cell-cycle regulated or do not follow closely in time, dark grey lines indicate protein interaction seen in yeast two-hybrid and protein complex assays, while medium grey lines indicate protein interactions seen only in yeast two-hybrid, and light grey lines show protein interaction seen only in protein complex. Proteins are shaded according to cell cycle phase during peak expression (G1, S, S/G2, G2/M, or M/1) from Spellman et al., Mol Bio Cell 9: 3273-3297 (1998).

FIG. 6. is a schematic illustration of an expression-anchored interaction network for sporulation. Black arrows: transcripts are expressed within 1 hour and proteins are connected directly by a physical interaction; grey arrows: transcripts are expressed within 1 hour and proteins are bridged by physical interactions with a third protein. The remaining lines are coloured as in FIG. 1. Proteins are colored according to sporulation phase assignment (metabolic, early I, early II, early-middle, middle, middle-late, or late).

FIG. 7. is a schematic illustration of the giant component containing 2229 proteins and 4000 pair-wise interactions. Interactions found using both Y2H and Co-IP screens are colored dark grey; Y2H only are colored medium grey; Co-IP only are colored light grey. Proteins have been shaded according to biological process (see Example 1).

### DETAILED DESCRIPTION OF THE INVENTION

A challenge to analyze a network of high-confidence interactions derived from experimental systems (e.g., yeast two-hybrid) is identifying the boundaries between distinct complexes. There is a finite limitation on the number of links one can follow from a central biological compound (e.g., a central protein) and still remain in the same complex or pathway. While progress has been made in identifying network motifs that describe short-range clustering (Lee et al., Science 298: 799-804 (2002); Milo et al; Science 298: 824-827 (2002)), it has been challenging to identify patterns over longer length scales. Described herein are methods using a predictive interaction model that includes one or more training sets. The methods of the present invention are sensitive to correlations over multiple protein-protein interaction links. The only input to the model is the proteomic data itself. Independent data sources, including experimental data from gene expression and genetic screens, tissue localization, and homology information, are reserved to validate the model predictions. The methods of the present invention are capable of predicting the correlation length of the network. In certain embodiments, the prediction may be validated if desired with comparison to independent data, such as experimental data. Further, knowledge of the correlation length may be used to develop algorithms that combine proteomics data with data obtained from genetic screens, extracting known and novel protein complexes with great selectivity, or with transcript profiles, providing a time-dependent view of network activity. The computational techniques disclosed herein have broad applicability given the increasing availability of genome-scale expression, genetic, and interaction data in model organisms and humans.

The present invention provides the merger of proteomics data with genetic and expression data. By choosing anchor proteins from genetic interactions, and including all proteins within a short radius of each anchor, subnetworks representing well-defined protein complexes are identified. Interactions between complexes indicate control points for pathways.

The combination of the temporal gene expression clustering with the static proteomic network topology according to the present method is a powerful, automated method for computationally extracting biologically relevant sub-networks. The methods of the invention have exceptional specificity, and resolve two problems of expression clustering: large clusters are automatically split into process-specific sub-clusters, and non-transcriptionally-regulated proteins are automatically included in the analysis. Further, less stringent criteria can capture an increased number of sub-networks.

As used herein “cellular interaction” includes any interaction between two or more biological compounds. Biological compounds include proteins, amino acids, nucleic acids, lipids, carbohydrates, cells, organelles, tissues, viruses and organisms. Statistical models are provided that assign a confidence score to every interaction. A “control cellular interaction” includes cellular interactions identified by means other than predictive interaction models. Control cellular interactions include experimentally-derived cellular interactions, such as cellular interactions identified by screening assays (e.g., Y2H or Co-IP).

Generation of Predictive Interaction Models using Confidence Scores: Prediction and Validation

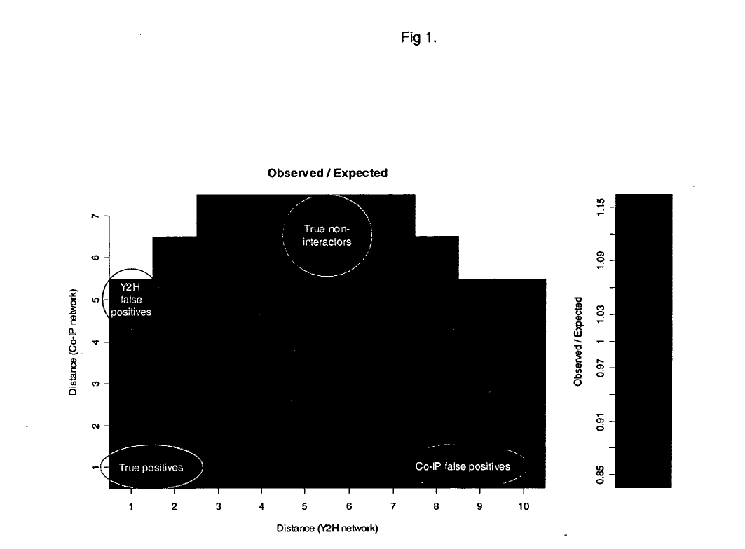

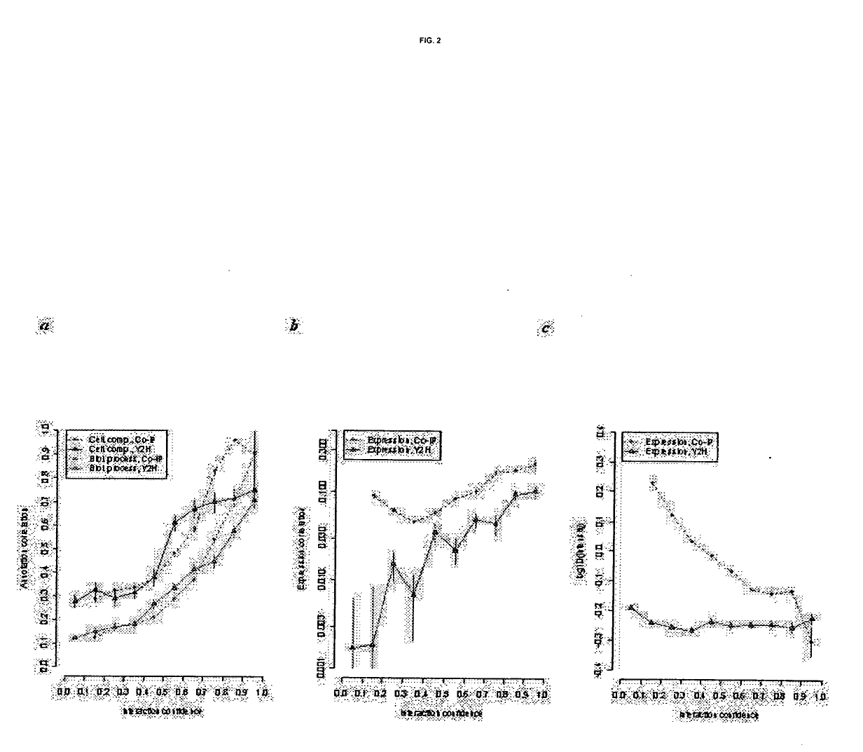

The present invention provides predictive interaction models that include the use of training sets. Training sets include instructive collections of data derived from two or more biological systems. By way of non-limiting example, training sets of positive and negative examples are generated by comparing protein networks built exclusively from published Y2H and Co-IP data (See Table S1). The networks are compared using a two-way contingency table in which pairs of proteins are binned according to their distance in the two networks (see Example 1). The ratio of interactions observed to interactions expected under the null hypothesis, no correlation between Y2H and Co-IP distances, is represented in FIG. 1 as a false-color map. As shown in FIG. 1, strong positive correlations are seen for pairs of proteins separated by 1-4 links in the Y2H network and 1-2 links in the Co-IP network, indicating that pairs separated by this number of links are likely enriched for biologically relevant interactions. These results indicate that each Co-IP link corresponds to approximately two Y2H links; thus, a protein-complex-like data set is obtained from the Y2H data set by grouping each protein with its nearest and second-nearest neighbors. Pairs of proteins that are directly connected in the Co-IP network and either one or two links apart in the Y2H network are selected as positive training set examples.

Inversely, pairs of proteins that are connected in one network but far-separated in the other network likely represent technology-specific spurious interactions. The distance comparison shows evidence for spurious interactions for Y2H data (red bin at Y2H distance 1, Co-IP distance 5) and for Co-IP data (red bin at Co-IP distance 1, Y2H distance 9). A negative training set is defined as Y2H interactions whose Co-IP distance is 3 or more, and Co-IP interactions whose Y2H distance is 5 or more. A third set of distances is also enhanced, protein pairs separated by 5-6 links in the Y2H network and 6-7 links in the Co-IP network. These pairs presumably represent proteins in unrelated pathways and could provide high-confidence examples of proteins that do not interact.

The training sets are used to build a predictive model that generated confidence scores for all interactions. Explanatory variables entered into the model represent the network connectedness in the neighborhood of a pair of proteins. A logistic regression model, similar to a Bayesian probabilistic network model, provides a convenient framework for inclusion of additional explanatory variables, such as co-expression and known annotations. These data sources are reserved as an independent validation of the interaction confidence scoring method. In some embodiments, data source is included as a possible explanatory variable.

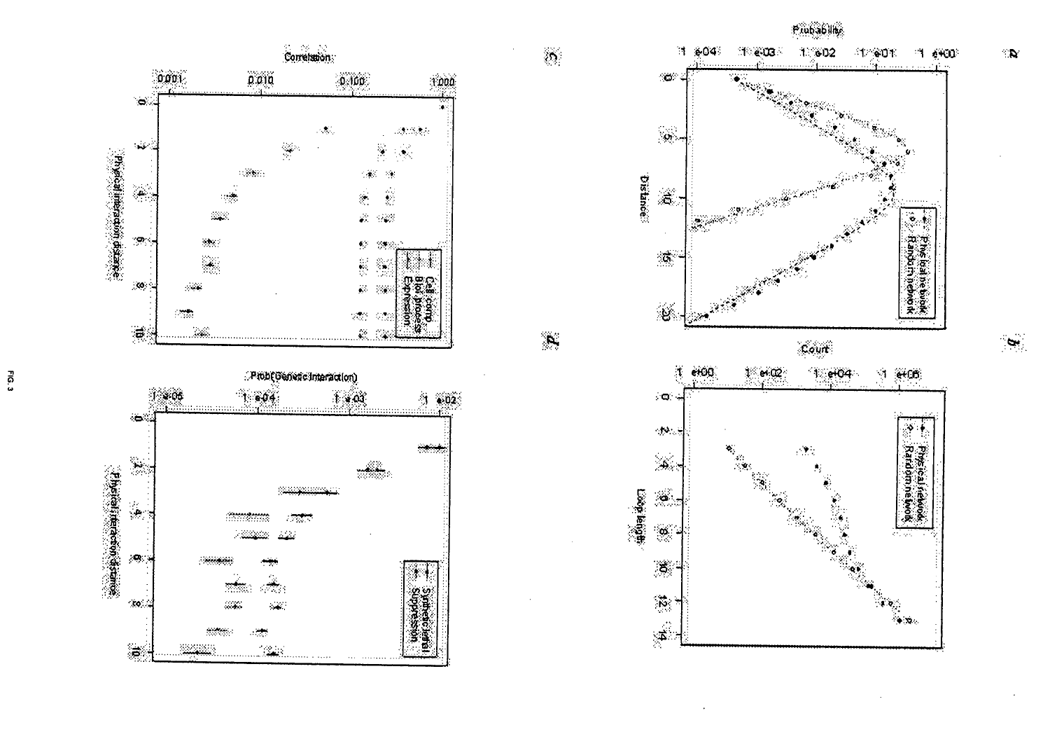

The confidence of an interaction between proteins A and B was highly correlated with the existence of bridging proteins, those connected to both A and B, and highly anti-correlated with the total number of interactions for a protein (supplementary table S2). For Y2H interactions, the Ito core, Uetz I, and Literature sources are strong positive predictors. The remaining Y2H data sources are not statistically significant predictors. The distinction between the two Co-IP data sources was not significant. Biological information was used to evaluate the validity of the derived interaction confidence scores. Correlations based on expression profiling (Hughes et al., Cell 102: 109-126 (2000)) and on cellular component and biological process annotations (Mewes et al., Nucleic Acids Research 30:31-34 (2002)) are calculated for each pair of proteins participating in an interaction. The interactions are then binned according to confidence score. Annotation correlations increase monotonically with confidence score, with a break in slope visible at a confidence score of 0.5 (FIG. 2a). High-confidence interactions are enriched for co-expressed genes (FIG. 2b). Interestingly, for Co-IP data, low-confidence interactions are also enriched for co-expressed genes. This latter effect was slightly more pronounced for the HMS-PCI data set. High co-expression for low-confidence interactions indicates that high levels of protein expression may contribute to weak or spurious interactions. In support of this conclusion, absolute expression level is inversely correlated with interaction confidence for Co-IP interactions, but not for Y2H interactions (FIG. 2c).

Network Correlations: Prediction and Validation

The low-confidence interactions (confidence score below 0.5) were removed, suppressed highly-connected hubs to focus on local network geometry (Tenenbaum et al., Science 290: 2319-2323 (2000); Roweis et al., Science 290: 2323-2326 (2000)), and extracted the giant connected component. The giant component retains the properties of a small-world network: clustering at short distances and random connectivity at longer scales. Properties of the giant component are displayed in FIG. 3. Proteins in the actual network have 9 degrees of separation on average, compared to 6 degrees of separation in a comparable random network (FIG. 3a). The actual network is flatter than a random network because clustering enhances local connections and suppresses far-away connections (Maslov et al., Science 296: 910-913 (2002)).

A topological statistic, the number of closed loops, that identifies the crossover length between clustering and random behavior, was introduced (FIG. 3b). The number of loops shows a significant enhancement over a random model up to loop length 9. Beyond this length, the distribution resembles that of a random network. The crossover near loop length 9 indicates that a non-trivial length scale of 4 links that characterizes the limit of biological coherence in the network: from a central protein, one can follow 4 links outward and 4 links back with an enhanced probability of remaining in the same cluster of interacting proteins. While the exact value of the crossover length depends in part on the methods used to suppress hubs, such a crossover is a reasonable selection criteria.

The interpretation that 4 links represents a correlation length is supported by results depicted in FIG. 3c, in which correlation coefficients based on annotation and co-expression have been averaged for pairs of proteins binned according to their shortest connecting path. Each correlation function shows a steep drop as the connecting path grows to 4 links. After 4 links, the decay is nearly complete as pairs have become random-like.

To again test biological coherence over 4 links, the probability of a genetic interaction between a pair of proteins was calculated as a function of the shortest path between the proteins on the physical network (FIG. 3d). This correlation also shows a steep decay to 4 links, followed by slow decay characteristic of a random network. Genetic interactions between near proteins might represent a physical interaction-based mechanism, while interactions between more distant proteins might represent a bypass or compensation mechanism.

Application: Combining Proteomics with Genetics

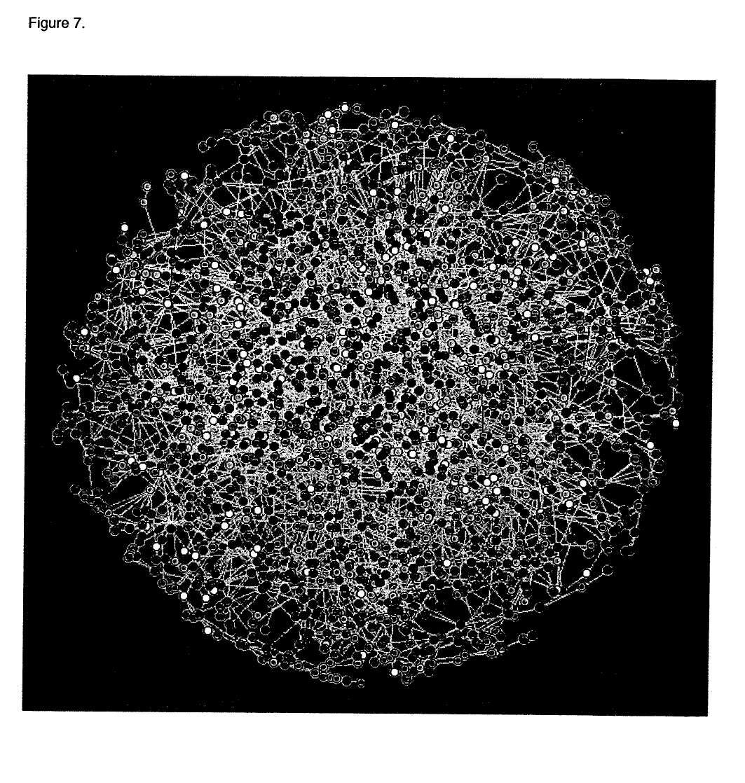

Despite the statistical assurance that interactions in the network are meaningful and the evidence that proteins connected by 4 or fewer links are likely to be functionally related, analysis of the network remains daunting due to the high connectivity (FIG. 7). Indeed, this static picture is essentially a compendium of all biological pathways involving over one-third of the yeast proteome. Further, recent studies make clear that static network topology is often insufficient to define function (Guet et al., Science 296: 1466-70 (2002); Bhalle et al., Science 297: 1018-1023 (2002)).

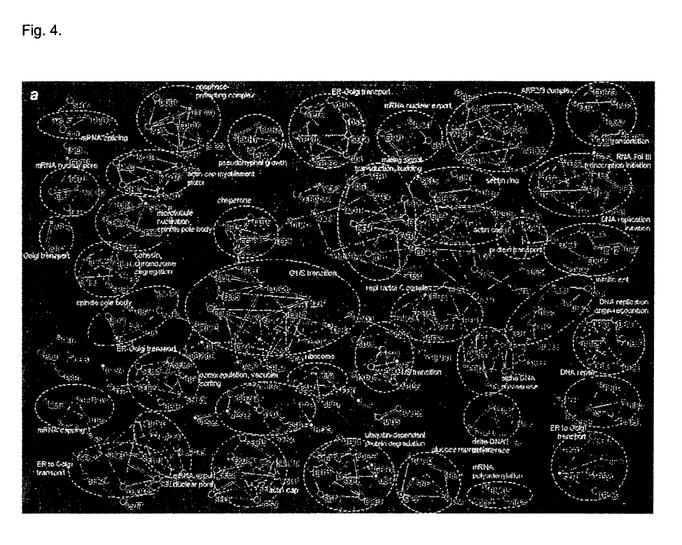

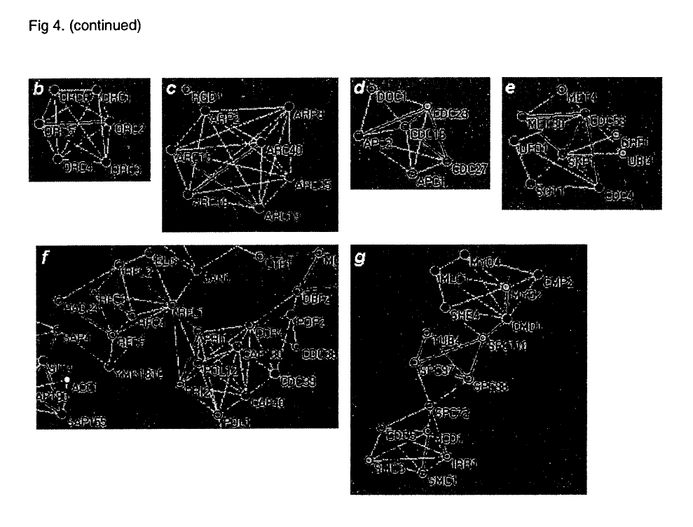

A correlation length of 4 links implies that 3 proteins, linked one to the next, are likely to have correlated function. To test this prediction, new sources of experimental data are included, e.g., western blotting, homology information, tissue expression. If the head and tail proteins are linked by independent functional information, for example by a genetic interaction, then a highly significant 4-membered loop is formed that should represent a single biological process. For example, FIG. 4a displays the result of such an analysis using proteins with known genetic interactions as anchor points for building subnetworks (see Example 1). The significance of this example is that the filtering is totally automated; given the genetic interaction anchors, protein complexes and pathways are efficiently determined.

Several well-defined complexes are immediately apparent (FIGS. 4b-e): the origin recognition complex (ORC1, ORC2, ORC3, ORC4, ORC5, ORC6); the actin related protein (ARP) complex (ARP2, ARP3, ARC15, ARC18, ARC19, ARC35, ARC40); the anaphase-promoting complex (APC1, APC2, CDC16, CDC23, CDC27, DOC1); and ubiquitin-dependent protein degradation machinery (CDC4, CDC53, MET30, UB14).

Beyond the automated detection of the well-characterized complexes above, our methods detect credible novel connections between complexes. These connections indicate that predictions concerning molecular mechanisms and control points underlying biological processes. In FIG. 4f, PRI1, PRI2, and POL12 of the alpha DNA polymerase/primase complex all have high-confidence physical interactions with RFC1, part of the DNA replication factor C complex, which performs leading strand elongation. The coupling of the primase complex to the RFC complex may serve a means to position the loading of PCNA and DNA polymerase delta at the appropriate location to initiate replication elongation following priming.

A second example of a series of related complexes (FIG. 4g) begins with the cohesin complex (SMC1, SMC3, IRR1, MCD1, and CDC5). MDC1, IRRI, and CDC5, a kinase essential for mitotic exit, all have physical interactions with SPC72, part of a spindle pole body complex (SPC72, SPC92, SPC98, SPC110, TUB4). In turn, SPC110 has physical interactions with CMDI, encoding calmodulin, and SHE4, required for asymmetric mating-type switching, which both have several interactions with myosin motor proteins (MYO2, MYO4, MLC1), and with CMP2/CNA2, encoding calcineurin.

Application: Combining Proteomics with Gene Expression

Functional genomics, the inference of gene function from transcript profiles, often proceeds through clustering of expression profiles to identify protein complexes and pathways. By merging transcription data with proteomics data, two problems with expression clustering are resolved: clusters of co-expressed transcripts that participate in disparate processes are split into process-specific sub-clusters, and proteins regulated by means other than transcription and invisible to transcript profiling are included in expression-anchored clusters.

The yeast mitotic cell cycle is an accepted prototypical model for eukaryotic cell division and proliferation. Its study has relevance for human disease as a model system for cancer. Comprehensive profiling of gene expression has shown that approximately 800 genes, or 10-15% of the yeast genome, are transcriptionally regulated during the cell cycle (Cho et al., Molecular Cell 2: 65-73 (1998); Spellman et al., Mol Bio Cell 9: 3273-3297 (1998)).

A similar analysis may be performed for the meiotic sporulation time course (Chu et al., Science 282: 699-705 (1998)), in which approximately 500 transcripts are found to be induced during sporulation. Within the cell cycle time course, each temporally regulated transcript has a single expression peak (Zhang et al., Proc Natl Acad Sci USA 98: 5631-5636 (2001)). Recent characterization of large-scale transcriptional regulatory networks in yeast provides an improved organizing framework for expression-based studies (Lee et al., Science 298: 799-804 (2002)).

This illustrates the two problems mentioned for expression clustering. First, a now-standard method for hierarchical expression-based clustering (Eisen at al. 1998) resulted in clusters with over 100 co-regulated transcripts. These clusters may then be manually analyzed with the aid of existing knowledge of the well-annotated yeast genome. While subdivision of large clusters into smaller, relevant networks can be accomplished in yeast because much is known, an automated method for subdividing expression clusters using other forms of genomic or proteomic information in any organism is advantageous. Second, clustering driven purely by expression neglects the large fraction of proteins whose transcripts are present constitutively but play an essential role. For example, cyclin-dependent kinases, key regulators of the cell cycle, are constitutively transcribed and therefore absent from any expression-driven approach. Interaction data allows pathways defined by expression anchors to be expanded to include the vast majority of proteins not regulated by the expression of their mRNA.

The static proteomic network is merged with the expression data from the mitotic cell division cycle by selecting as anchors pairs of proteins satisfying one or more of the following criteria:

1. Each protein is temporally regulated as defined by, e.g., Spellman et al. (800 proteins).

2. Both proteins are present in the static high-confidence physical network (443 proteins).

3. The time delay in peak transcription for the pair is no greater than 10% of the total cell cycle period.

4. The proteins are either connected directly or are bridged by a single protein in the physical network (118 proteins, 141 time-directed interactions).

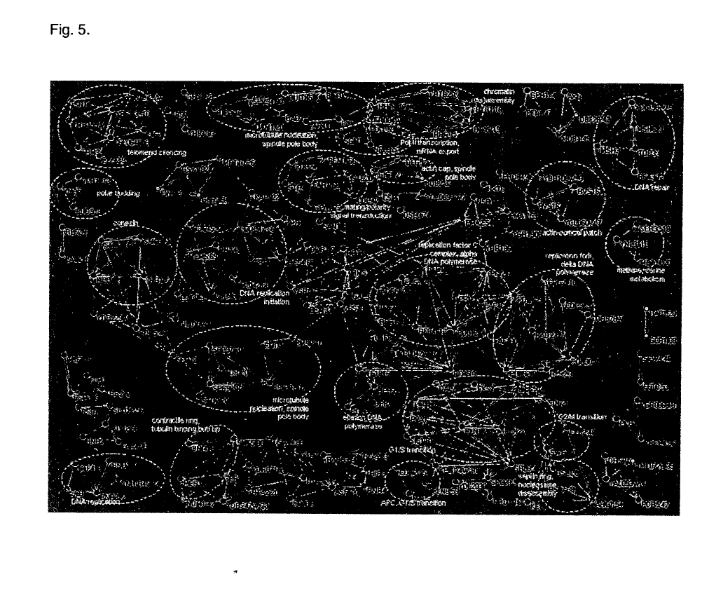

These anchoring pairs and all proteins directly connected to at least two anchors are extracted from the static network to form a network containing 201 proteins with 141 time-directed interactions and 283 additional physical interactions (see, e.g., FIG. 5).

By this method, sub-networks with protein ordered by peak mRNA expression and their interaction partners are easily identified. These sub-networks are illustrated in Supplementary Table S1. These fully automated predictions are in good concordance with existing knowledge of the yeast cell division cycle.

Notable among the complexes identified by this automated approach is the cyclin-dependent kinase complex, several complexes involved in DNA repair and replication, a spindle pole body microtubule nucleation complex, and the cohesin complex.

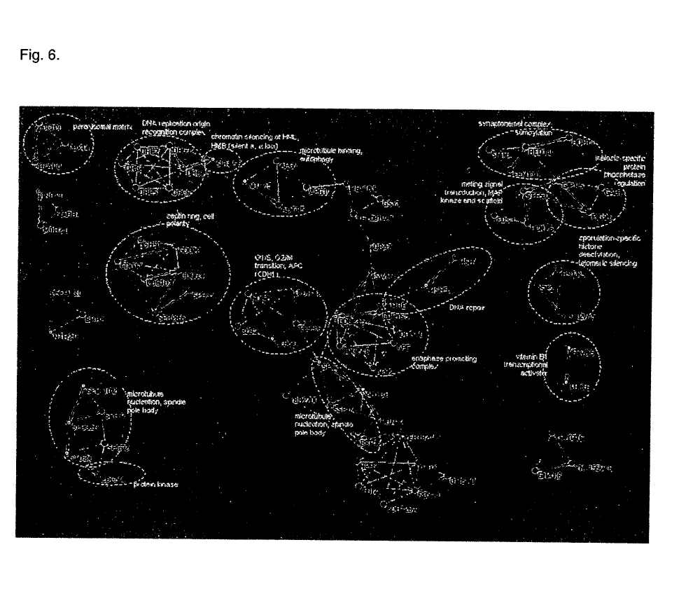

A similar analysis is performed for the gene expression timecourse of sporulation. The time of expression for each transcript is defined as the time to reach half-maximum log-scale expression. Pairs of induced proteins either directly connected or bridged by a single protein and with at most an hour difference in characteristic expression time are selected as anchors. Analysis was performed as described above for the cell cycle data. Results are presented in FIG. 6.

Some of the sub-networks produced by this analysis are similar to those produced using the cell cycle timecourse, and sporulation-specific sub-networks are also provided. The cyclin-dependent kinase complex is identified as an essential component to both processes. Three complexes that are readily observed in the sporulation timecourse data include the origin recognition complex (ORC), the autophagy complex and the septin ring complex. While each of these complexes is present in mitotically growing cells, it is likely that the timing information from the sporulation expression timecourse allows these complexes to be extracted. Thus, the use of diverse sets of expression data allows a more comprehensive extraction of biological sub-networks.

Analysis of protein physical interaction networks has often been hindered by lack of confidence in the credibility of the individual interactions composing a network. As described herein, it is now possible to define a quantitative confidence measure based entirely on screening statistics and network topology. The principal assumption underlying the confidence measure is that non-specific interactions are likely to be technology-specific. The use of explanatory variables including additional information such as expression correlation or annotation, provides an improved measure of interaction confidence.

The yeast high-confidence network described herein is highly complex, essentially a superposition of all the pathways involving protein interactions for over one-third of the yeast proteome. The topological properties of the resulting high-confidence network indicate a correlation length of 4 links, supporting the assertion that biological coherence also extends over approximately 4 links. Comparison with annotations and expression measurements shows a consistent extent of biological coherence, suggesting a relationship to the diameter of a typical protein complex.

The following examples are intended only to illustrate the present invention and should in no way be construed as limiting the subject invention.

### EXAMPLE 1

Data sources. A summary of each physical interaction data source is provided in supplementary table Si. For each data source, the exponent a from the power-law fit N(J)=N₀J^{α}, where N(J) is the number of proteins connected to exactly J other proteins, was calculated and appears in the table. Large negative values of α may indicate more stringently filtered data. The column labeled “All” refers to interactions involving a pair of proteins that is resolved uniquely in the Saccharomyces Genome Database. The columns “High-conf”, “Hub-free”, and “Giant” refer to the subset of interactions included in the high-confidence, hub-free, and final giant connected component networks, respectively.

Y2H data. Y2H data sources are termed Uetz I (Uetz et al., Nature 403: 623 (2000)), Uetz II (Uetz and Fields 2002 New two-hybrid interactions generated since our last survey was published. Available on Stan Fields' web site at the University of Washington.), Ito core and Ito full (Ito et al., Proc Natl Acad Sci USA 98: 4569-4574 (2001)), Tong (Tong et al., Science 295: 321-324 (2002)), and MIPS (Mewes et al., Nucleic Acids Research 30:31-34 (2002)). An additional category, Lit(erature), was assigned to 620 interactions appearing in the MIPS data but not in Uetz I. Because not all data sets indicated which protein was the bait and which was the prey, the interactions are treated as undirected. The full Y2H data set contained 3941 proteins and 6395 interactions, of which 3660 proteins and 6239 interactions are in a giant connected component. Distances between proteins in the giant component are calculated and saved for network self-validation.

Co-IP data. Co-IP data sources are termed Gavin (Gavin et al., Nature 415: 141-147 (2002)) and Ho (Ho Y et al., Nature 415: 180-183 (2002)). Complexes consisted of a bait proteins and the hits that are isolated in a complex with it. From the authors' pre-filtered data sets downloaded from supplementary data, pairwise interactions are inferred between all proteins within each complex. The power-law decay of the interaction count distribution is slower for the Co-IP data than for Y2H interactions, indicating the possibility of a greater number of non-specific interactions in the Co-IP data. The full Co-IP data set contained 2271 proteins and 41,775 interactions, of which 2211 proteins and 41,707 interactions are in the giant connected component. Distances between proteins in the giant component are calculated and saved for network self-validation.

Network self-validation. Because the Y2H and Co-IP screens are both incomplete due to insufficient sampling and technical limitations, not every interaction that appears in one is expected to appear in the other. Nevertheless, proteins connected by a short path of Y2H interactions should also be connected by a short path of connections inferred from Co-IP data. The distance between a pair of proteins was calculated in each network, and the distances are cross-tabulated as N_{obs} (D_{y},D_{c})=the number of pairs present in both networks with distance D_{y} in the Y2H network and distance D_{c} in the Co-IP network. The corresponding marginal counts N_{Obs} (D_{y}) and N_{Obs} (D_{c}) are also tabulated, with

Nₜₒₜ=Σ_{D}_{_{y}}_{, D}_{_{c}}N_{obs}(D_{y}, D_{c})=Σ_{D}_{_{y}}N_{obs}(D_{y})=Σ_{D}_{_{c}}N_{obs}(D_{c}).

The most likely values of D_{y} and D_{c} are 5 and 3, respectively. The expected number of pairs Nₑₓₚ(D_{y},D_{c}) under the null hypothesis that distances in the two networks are independent was calculated as Nₑₓₚ (D_{y},D_{c})=N_{obs} (D_{y})N_{obs} (D_{c})/Nₜₒₜ. The ratio of the observed to expected number of counts, N_{obs} (D_{y},D_{c})/Nₑₓₚ (D_{y},D_{c}), is shown in FIG. 1 as a false-color map. Under the null hypothesis, N_{obs} (D_{y},D_{c}) should follow a Poisson distribution with mean Nₑₓₚ (D_{y},D_{c}). Two-sided p-values show significant deviations from the null hypothesis: 30 of the 62 bins depicted in FIG. 1 are significant at p=0.01, 23 are significant at p=0.001, and 17 are significant at p=0.0001.

For Y2H interactions (D_{y}=1), the leftmost column of FIG. 1 represents the distribution of distances D_{c} for each corresponding pair of proteins in the Co-IP network. For D_{c}=1, the training score for a predictive model was set to 1 (367 examples); for D_{c}=3 or more, the training score was set to 0 (765 examples); the remaining 5263 pairs of proteins are not used for training. Several explanatory variables are defined to enter into a logistic regression model for Y2H interaction confidence. For each protein i, the total number of Y2H interactions nᵢ including that protein are calculated. Then, for an interaction between a pair of proteins arbitrarily labeled 1 and 2, define nₘᵢₙ=min(n₁,n₂); nₘₐₓ=max(n₁,n₂); n_{geom}=(n₁n₂)^{1/2}; n₁₂=the number of proteins interacting with both protein 1 and protein 2; and the Jaccard coefficient jac=n₁₂/(n₁+n₂−n₁₂). Mathematical transforms, primarily sqrt and log, of these variables, are also entered. Indicator variables are defined as 1 or 0 if an interaction was found in Uetz i, Uetz II, Ito core, Ito full, Tong, or Lit. Positive and negative training examples are weighted inversely to their proportion in the training set, which helps ensure that a logistic response of 0.5 is an appropriate separation between high-confidence (≧0.5) and low-confidence (<0.5) interactions. R is an open source integrated suite of software facilities for data manipulation, calculation and graphical display provided by the R Foundation for Statistical Computing (see their web site at r-project-org). R provides a wide variety of statistical (e.g., linear and nonlinear modelling, classical statistical tests, time-series analysis, classification, clustering) and graphical techniques. The glm function of R version 1.3.1 is used to perform the logistic regression (binomial link, weights as stated, all other parameters, including Akaike information criterion for stepwise model selection). The final model includes just [In(nₘₐₓ)]², (n₁₂)^{1/2}, Uetz I, Ito full, and Lit as significant explanatory variables (see supplementary table 2A). Using 0.5 as the threshold for a high-confidence interaction, 281 of the 367 positive examples are correctly predicted (23% false-negative rate), and 572 of the 765 negative examples are correctly predicted (34% false-positive rate). The logistic regression model performed better than other types of models examined, including linear discriminant analysis, support vector machines, and decision trees.

For interactions inferred from Co-IP data (D_{c}=1), the bottom row of FIG. 1 represents the distribution of distances D_{y} for the corresponding pair of proteins in the Y2H network. For D_{y}=1 or 2, the training score for a predictive model was set to 1 (917 examples); for D_{y}≧5, the training score was set to 0 (9376 examples). Positive and negative examples are weighted inversely according to their fraction of the training set. A re-calculation from the Co-IP data the interaction was performed, counts nᵢ for each protein and, for an interaction between proteins labeled 1 and 2, the explanatory variables nₘᵢₙ, nₘₐₓ, n₁₂, and jac as defined above. Indicator variables for the data source (Gavin, Ho) are also included. While an explicit indicator for whether an interaction was bait-hit or hit-hit was is not provided (Bader et al., Nature Biotech 20: 991-997 (2002)), the jac variable includes this information implicitly. Stepwise regression was performed as above and the three terms In(n_{geom}), In(n₁₂), and In(jac) are found to be significant (Table S2B). For the training set, 554 of 917 positive examples are correctly predicted (40% false-negative rate) and 6763 of the 9376 negative examples are correctly predicted (28% false-positive rate). Interactions identified experimentally by Y2H and Co-IP methods may occur between proteins that are present in a stable complex but that do not have a direct physical interaction.

Confidence score assessment. Confidence scores were assessed by comparison with biological annotations and with expression data. Annotations for biological process (subcellular localisation; metabolism; transcription; cell cycle and DNA processing; cellular transport and transport mechanisms; transport facilitation; protein fate; cell fate; protein synthesis; cell rescue, defense and virulence; energy; control of cellular organization; regulation of or interaction with cellular environment; transposable elements, viral and plasmid proteins; cellular communication/signal transduction mechanism; protein activity regulation; protein with binding function or cofactor requirement) and cellular component (nucleus; cytoplasm; mitochondria; plasma membrane; ER; cytoskeleton; golgi; vacuole; transport vesicles; peroxisome; cell wall; extracellular; endosome; lipid particles; microsomes) were obtained from the corresponding MIPS functional classification and subcellular localization catalogs (Mewes et al., Nucleic Acids Research 30:31-34 (2002)). Annotation correlations for each pair of proteins were calculated separately for the biological process and cellular component classification.

A typical approach to calculating an annotation-based correlation for a pair of proteins has been to define the correlation as 1 if their top-level annotations were identical and 0 otherwise. Because many proteins are placed in multiple top-level categories, however, this would require manual re-assignment of each such protein to a single top-level category. Instead, the Jaccard correlation coefficient was used, defined as the number of top-level annotations shared by a pair of proteins divided by the number of unique top-level annotations for both proteins. Results from the Jaccard correlations did not differ markedly from results using a 1/0 correlation (1 if all the top-level annotations match, 0 otherwise) or from a Pearson correlation between vectors of 1's and 0's corresponding to presence or absence of each possible top-level annotation

Annotation and expression correlations were calculated for each interaction. Next, interactions were binned according to confidence score (bin width 0.1), and the mean and standard error of the mean for each bin was calculated. Results are displayed in FIG. 2. The annotation correlation should correspond roughly to the true-positive rate for a given confidence score.

Expression data for 300 experimental conditions were obtained from Hughes et al., Cell 102: 109-126 (2000). Pearson correlation coefficients for log-ratio measurements were calculated for all pairs of interacting proteins having 290 or more pair-wise measurements. The absolute expression level for each interaction was calculated as the mean of the absolute levels of the two proteins. These levels were in turn calculated as the mean of the log₁₀(intensity) values for the corresponding transcripts. The p-value for a negative slope of the linear regression line was 1.1×10⁻⁵ for the Co-IP data and 0.24 for the Y2H data.

Final combined network. Y2H and Co-IP interactions with a confidence scores 0.5 or greater were combined into a single network. Hubs were suppressed to give greater emphasis to local connectivity (Tenenbaum et al., Science 290: 2319-2323 (2000); Roweis et al., Science 290: 2323-2326 (2000)). First, the number of interactions is calculated for each protein, and the 46 proteins with over 40 interactions were removed from the network (1.5% of the total number of proteins, 10.5% of the edges). Next, the number of interactions ni for each protein was re-calculated, the edge product ninj for each interaction between proteins i and j was calculated, and interactions with an edge product greater than 120 were deleted from the network (12% of the remaining proteins, 66.0% of the remaining edges). The overall features of the network connectivity described below, in particular a well-defined crossover point between local clustering and long-range random connectivity, were observed for a broad range of pruning parameters.

The giant connected component, containing 2229 proteins and 4000 interactions, was extracted and the distances between all pairs of proteins in the giant component and the number of closed loops (interactions between proteins A and B, B and C, . . . , Z and A, with no protein repeated) were calculated (FIGS. 3A,B). Annotation and expression correlations, as previously defined, were binned as a function of distance in the physical network for analysis (FIG. 3C). The probability of a genetic interaction was also calculated as a function of the distance between proteins (FIG. 3D, see below for the source of genetics interactions).

Randomized network. Descriptive statistics were also calculated for randomized networks generated to conserve the empirical distribution of interactions per protein. Using a standard approach, pairs of interactions A-X and B-Y were swapped to yield a new network with interactions A-Y and B-X. Swaps creating a self-connection or multiple connections between a pair of proteins resulting in a disconnected network, or that created an edge with edge product greater than 120 were rejected (approximately 0.5% of swaps were rejected). Trial swaps were generated by listing the interactions in random order, with each interaction appearing twice in the list (forward and backward). This list was combined with a second random permutation of interactions, and each pair of interactions was then considered in turn. This procedure was employed 4 times, resulting in 8 attempted swaps for each interaction. Statistics were then averaged over an ensemble of twenty simulated random networks.

The following formulations, for which general comments are provided, were applied in the analysis of the data collected in the making of the present invention Distance distribution in a random network (mathematical model). In a random network with N proteins and J interactions per protein, the distance distribution for pairs of proteins can be estimated from an approximate recursion formula for the number of proteins Wₙ within n links of a central protein,

Wₙ≈Wₙ₋₁+Wₙ₋₁J·[1−(Wₙ₋₁/N)].

The term Wₙ₋₁ is the number of proteins within n-1 links. Each of these is connected to J other proteins, which add to the total at step n. The term in square brackets is the probability that one of the new proteins was already included in the count. An approximation is made that most of the density of the growing ball is at the outer shell, which yields an equation for logistic growth. Defining xₙ as Wₙ/N, changing the discrete index n to a continuous coordinate t, and passing from a difference equation to a differential equation yields a Bernoulli equation

\dot{x}(t)=−Jx(t)[1−x(t)],

that is solved using the standard substitution y=(1/x)−1. The solution for the continuous coordinate is

x(t)−1/[1+N exp(−Jt)].

When a difference equation is converted to a differential equation, it is typical that geometric growth becomes exponential growth (as in the equations for compound interest). To obtain a function appropriate for the original difference equation, the exponential is converted back to a geometric term,

x(t)=1/[1+N(1nJ)J⁻¹].

The probability pₙ that two proteins are exactly n links apart is then \dot{x}(t) evaluated at t=n,

pₙ=NJⁿ/[N+(Jⁿ/1nJ)]².

The most-likely separation ñ satisfies J^{ñ}=N1nJ.

Closed loops (mathematical model). In a network with N proteins, the number of possible closed loops of n proteins is [N!/(N−n)!]/2n≈N/2n . The numerator is the number of ways of selecting n proteins in order without replacement; the denominator corrects for the symmetry of closed loops (n equivalent starting positions, forward-backward traversal). The approximation, corresponding to selection with replacement, is highly accurate when the number of proteins in the loop is small compared with the total number of proteins.

With J interactions per protein, the probability that two proteins are connected by an interaction in the random model is J/N; the probability that all n interactions are present is (J/N)ⁿ. The expected number of closed loops of length n is therefore Jⁿ/2n. Note that the number of closed loops of length n depends only on J, not on the total number of proteins N, provided n<<N.

The ratio of the number of closed loops of length 3 to the number expected in a random network, J³/6 where J is the number of interactions per protein, is typically defined as the clustering coefficient for a small-world network.

Genetic interactions. A total of 794 synthetic lethal interactions and 360 suppression interactions were obtained by combining literature data (MIPS) and the results of a high-throughput screen (Tong et al., Science 295: 321-324 (2002)); 30 interactions appeared in both categories.

Visualization. Network visualization was performed using Pajek (Batagelj and Mrvar 1998) using Kamada-Kawai 2D layout. Protein colors were selected according to MIPS biological process annotation: Cell cycle=Cyan; Cell defense=Yellow; Cell environment=Tan; Cell fate=Red; Cell organization=Yellow; Energy=Pink; Metabolism=White; Protein fate=Orange; Protein regulation=Purple; Protein synthesis=CadetBlue; Signal transduction=TealBlue; Transcription=LimeGreen; Transport=Maroon.

Extraction of anchored networks. Anchored networks were extracted by first identifying pairs of proteins having a genetic interactions and either connected directly or bridged by one or two links on the physical interaction network. The nearest neighbors of these anchor proteins were then retained providing they had interactions with at least two anchored proteins.

Computational sampling was employed to calculate the statistical significance of the extracted networks (Ideker et al., Bioinformatics 18: S233-S240 (2002)).

TABLE S1
Physical interaction data sources

| | | Number of | | | | Number of | | | |
|---|---|---|---|---|---|---|---|---|---|
| | | proteins | | | | interactions | | | |
| | | | High- | Hub- | | | High- | Hub- | |
| Source | Power-law | All | conf | free | Giant | All | conf | free | Giant |
| Uetz I | −2.4 ± 0.1 | 974 | 882 | 770 | 668 | 893 | 747 | 598 | 537 |
| Uetz II | −1.7 ± 0.1 | 484 | 81 | 61 | 61 | 505 | 78 | 53 | 53 |
| Ito core | −1.8 ± 0.2 | 780 | 673 | 611 | 522 | 747 | 569 | 469 | 417 |
| Ito full | −1.4 ± 0.1 | 3253 | 801 | 704 | 599 | 4,358 | 703 | 564 | 503 |
| Tong | −1.3 ± 0.2 | 142 | 32 | 25 | 25 | 229 | 51 | 20 | 20 |
| MIPS | −2.2 ± 0.1 | 1556 | 1259 | 1077 | 927 | 1,669 | 1,227 | 922 | 831 |
| All Y2H | | 3941 | 1646 | 1451 | 1219 | 6,395 | 1,754 | 1376 | 1233 |
| Gavin | −1.16 ± 0.05 | 1351 | 1304 | 889 | 803 | 16,978 | 7,412 | 1439 | 1332 |
| Ho | −1.12 ± 0.04 | 1569 | 1486 | 1136 | 1074 | 26,308 | 6,496 | 1836 | 1788 |
| All Co-IP | | 2271 | 2210 | 1726 | 1583 | 41,775 | 12,645 | 3083 | 2930 |
| Y2H + Co-IP | | 4627 | 2985 | 2585 | 2229 | 47,783 | 14,104 | 4288 | 4000 |

TABLE S2
Confidence score model parameters
Table S2A. Y2H interactions

glm(formula = expert ˜ log(nmax)\hat{ }2 + sqrt(n12) + u1 + ic + lit,
family = binomial, data = small, weights = weights)
Deviance Residuals:

| Min | 1Q | Median | 3Q | Max |
|---|---|---|---|---|
| −1.6316 | −0.5655 | −0.3364 | 0.4404 | 2.1043 |

Coefficients:
Estimate Std. Error z value Pr(>|z|)

| | | | | |
|---|---|---|---|---|
| (Intercept) | −0.3905 | 0.2278 | −1.715 | 0.086393. |
| log(nmax)\hat{ }2 | −0.1238 | 0.0217 | −5.705 | 1.16e−08*** |
| sqrt(n12) | 1.1322 | 0.1916 | 5.908 | 3.46e−09*** |
| u1 | 1.2296 | 0.2783 | 4.419 | 9.91e−06*** |
| ic | 1.9694 | 0.3372 | 5.841 | 5.20e−09*** |
| lit | 1.0556 | 0.2765 | 3.818 | 0.000135*** |

Signif. codes: 0 ‘***’ 0.001 ‘**’ 0.01 ‘*’ 0.05 ‘.’ 0.1 ‘ ’ 1
(Dispersion parameter for binomial family taken to be 1)
Null deviance: 687.65 on 1131 degrees of freedom
Residual deviance: 512.17 on 1126 degrees of freedom
AIC: 387.02
Number of Fisher Scoring iterations: 4

Table S2B. Co-IP interactions

glm(formula = expert ˜ log(ngeom) + log(n12) + log(jac),
family = binomial,
data = small, weights = weights)
Deviance Residuals:

| | | | | |
|---|---|---|---|---|
| Min | 1Q | Median | 3Q | Max |
| −0.6626 | −0.3561 | −0.2890 | −0.2367 | 1.8028 |

Coefficients:
Estimate Std. Error z value Pr(>|z|)

| | | | | |
|---|---|---|---|---|
| (Intercept) | 1.2735 | 0.1788 | 7.123 | 1.06e−12*** |
| log(ngeom) | −0.5047 | 0.2555 | −1.975 | 0.048213* |
| log(n12) | 0.9870 | 0.2915 | 3.386 | 0.000709*** |
| log(jac) | 0.3661 | 0.2074 | 1.765 | 0.077590. |

Signif. codes: 0 ‘***’ 0.001 ‘**’ 0.01 ‘*’ 0.05 ‘.’ 0.1 ‘ ’ 1
(Dispersion parameter for binomial family taken to be 1)
Null deviance: 2316.0 on 10292 degrees of freedom
Residual deviance: 2047.6 on 10289 degrees of freedom
AIC: 1144.1
Number of Fisher Scoring iterations: 3

TABLE S5
Cell division cycle sub-networks

Polar budding (VPH1, BUD9)
Components of Set3C complex (SNT1, SIF2) and ZDS2, involved in telomeric silencing
Cohesin complex (SMC1, SMC3, IRR1, MCD1)
microtubule nucleation and spindle pole body structure (SPC72, SPC97, SPC98, TUB4, STU2, SPC110)
DNA replication initiation (CDC45, CDC46, CDC47, CDC54, DNA43, MCM6)
DNA replication (RNR1, CTF4, CCE1, RNR3)
The cyclin-dependent kinase complex (CDC28, CLB3, CLB5, CKS1, DAL7, PCA1, SIC1, CLN1, CLN2,
CLN3) connected to anaphase promoting complex (CDC20, MAD3) by interactions between CLN3 and
MAD3
Main G1/S checkpoint also connected to G2/M checkpoint (CLB2, SWE1) by interactions between CDC28,
CLB5, and CLB2.
G2/M checkpoint connected to septin ring and nucleosome disassembly (HSL1, HSL7, AHC1)
Replication factor A complex and alpha DNA polymerase (leading strand synthesis), replication fork delta
DNA polymerase, and epsilon DNA polymerase (lagging strand synthesis) all observed with multiple
interactions between each complex.
Actin cortical patch (RVS161, RVS167)
Mating/polarity signal transduction (BEM1, CDC24, FAR1, STE20, CDC42, STE4, GPA1)
Microtubule nucleation at spindle pole body (TID3, SPC34, DAM1, DAD1, DUO1, LAP4, BIM1, BIK1)
connected to chromatin (dis)assembly (HHT1, HTB1, HTB2, HTA1, HHF1, SPT6) by interactions between
BMI1 and HHT1.
Chromatic (dis)assembly complex also connected to Pol II transcription and mRNA export (PDI1, NPL3,
SGN1) by interactions between YER084W and PDI1

TABLE S6
Sporulation sub-networks

Septin ring and polarity (CDC3, CDC10, CDC11, CDC12, SHS1, SPR28, SPR3)
Microtubule nucleation and spindle pole body (SPC110, SPC97, SPC98, SPC72, STU2), connecting to a
protein kinase (CDC5)
DNA repair (MSH4, MSH5, MRE11) connecting to an anaphase promoting complex (APC1, APC2, CDC16,
CDC23, CDC27, DOC1) which in turn connects to G1/S and G2/M checkpoint proteins (CLB3, CLB5, CDH1,
CDC28, CLB2). CLB5 interacts physically with SPC29, part of the spindle pole body complex involved in
microtubule nucleation (SPC29, SSP1, BBP1)
The DNA replication origin recognition complex (ORC1 through ORC6), which interacts with SIR1 (chromatin
silencing at the HML and HMR loci, silenced duplicates of the a and α genes)
Mating signal transduction: MAP kinase (FUS3) and MAP kinase scaffold protein (STE5) connected to
meiosis-inhibiting protein kinase (RCK1). The scaffold protein also interacts with meiosis-specific regulators
of protein phosphatase 1 (GIP1, PIG2, GAC1). GIP1 interacts with proteins that are part of the
synaptonemal complex (RED1, HOP1), which interact in turn with the yeast homolog of SUMO (SMT3) and a
protein thought to be responsible for chromatin maintenance (EMC11).
Sporulation-specific histone deacetylation (HST1) and telomeric silencing (SIF2)

The invention now being fully described, it will be apparent to one of ordinary skill in the art that many changes and modifications can be made thereto without departing from the spirit or scope of the invention and the appended claims. Those skilled in the art will recognize, or be able to ascertain using no more than routine experimentation, numerous equivalents to the specific procedures described herein. Such equivalents are considered to be within the scope of the present invention and are covered by the following claims. All publications, patent applications, patents, and other references mentioned herein are incorporated by reference in their entirety. In case of conflict, the present specification, including definitions, will control. In addition, the materials, methods, and examples are illustrative only and not intended to be limiting. The appropriate components, processes, and methods of those patents, applications and other documents is selected for the present invention and embodiments thereof.

What is claimed is:

1. A method for identifying a cellular interaction in a biological system, the method comprising:
a) providing a predictive interaction model, wherein said predictive interaction model comprises one or more training sets; and
b) applying said predictive interaction model to said biological system to identify a high-confidence interaction,
thereby identifying one or more cellular interactions in said biological system.

2. A method of claim 1, further comprising validating said identified cellular interaction, said validation comprising comparing said identified cellular interaction with a control cellular interaction.

3. A method of claim 2, wherein said control cellular interaction is an experimentally derived cellular interaction.

4. A method of claim 3, wherein said experimentally derived cellular interaction is identified using a yeast two-hybrid system or a co-immunoprecipitation system.

5. The method of claim 1, wherein said one or more training sets are selected from the group consisting of postive training sets, negative training sets, and non-interacting training sets, or a combination thereof.

6. A method of claim 1, wherein said predictive interaction model further comprises one or more explanatory variables.

7. The method of claim 6, wherein said explanatory variable is selected from the group consisting of co-expression of nucleic acids, sequence similarity of polypeptides, and domain similarity of polypeptides.

8. The method of claim 1, wherein said cellular interaction is a protein-protein interaction.

9. The method of claim 1, wherein said cellular interaction is a protein-nucleic acid interaction.

10. A method of identifying an interacting protein that interacts with a test protein in a biological system, the method comprising:
a) providing a predictive interaction model, wherein said predictive interaction model comprises one or more training sets; and
b) applying said predictive interaction model to said biological system to identify a high-confidence interaction between said test protein and said interacting protein,
thereby identifying one or more interacting proteins.

11. The method of claim 10, wherein said interaction is validated using a yeast two-hybrid system or a co-immunoprecipitation system.

12. The polypeptide identified by the method of claim 9.

13. A method of identifying a compound that modulates a cellular pathway in a biological system, the method comprising contacting said biological system with a candidate agent, such that a high-confidence interaction between a test protein and an interacting protein is modulated,
thereby identifying a compound that modulates said cellular pathway in said biological system.

14. The method of claim 13, wherein said agent increases the expression of said test protein.

15. The method of claim 13, wherein said agent decreases the expression of said test protein.

16. The method of claim 13, wherein said agent increases the expression of said interacting protein.

17. The method of claim 13, wherein said agent decreases the expression of said interacting protein.

18. The compound identified by the method of claim 13.

19. A method of claim 13, wherein said biological system comprises a transgenic animal.

20. The method of claim 19, wherein said transgenic animal is a transgenic mouse.

21. A method of identifying a compound that modulates a cellular pathway in a biological system, the method comprising:
a) providing a predictive interaction model, wherein said predictive interaction model comprises one or more training sets;
b) applying said predictive interaction model to said biological system to identify a high-confidence interaction between said test protein and said interacting protein;
c) contacting said biological system with a candidate agent, such that said high-confidence interaction between said test protein and said interacting protein is modulated,
thereby identifying a compound that modulates said cellular pathway in said biological system.

22. A method of diagnosing a test subject affected by an aberrant cellular pathway, the method comprising:
a) providing a predictive interaction model, wherein said predictive interaction model comprises one or more training sets;
b) applying said predictive interaction model to a biological sample derived from said test subject to identify a first high-confidence interaction;
c) comparing said first high-confidence interaction with a second high-confidence interaction derived from a biological sample from a reference subject, wherein said reference subject is not affected by an aberrant cellular pathway,
whereby a difference in said first high-confidence interaction from said second high-confidence interaction indicates that said test subject is affected by an aberrant cellular pathway.

23. A method of diagnosing a test subject suffering from or is at risk of disease or disorder characterized by an aberrant cellular pathway, the method comprising:
a) providing a predictive interaction model, wherein said predictive interaction model comprises one or more training sets;
b) applying said predictive interaction model to a biological sample derived from said test subject to identify a first high-confidence interaction;
c) comparing said first high-confidence interaction with a second high-confidence interaction derived from a biological sample from a reference subject, wherein said reference subject is not suffering from or at risk of disease or disorder,
whereby a difference in said first high-confidence interaction from said second high-confidence interaction indicates that said test subject suffers from or is at risk of disease or disorder.

24. The method of claim 23, wherein said disease is selected from the group consisting of a cell proliferation-associated disease, a cell differentiation-associated disease, and an apoptosis-associated disease.

25. A database comprising a predictive interaction model, wherein said predictive interaction model comprises one or more training sets.
